(19) United States
(12) Patent Application Publication
(10) Pub. No.: US 2024/0397921 A1
(43) Pub. Date: Dec. 5, 2024

(54) CAGE FOR THE LARGE-SCALE PRODUCTION OF INSECT EGGS DEFINING A CONFINED BOX-SHAPED SPACE FOR THE INSECTS

(71) Applicant: INNOVAFEED, NESLE (FR)

(72) Inventors: Bastien Oggeri, Nesle (FR); Xavier Daussin, Nesle (FR); Gustavo Krapf, Nesle (FR); Géraldine Schuster, Nesle (FR); Emmanuel Mernier, Nesle (FR); Oliver Vidal, Nesle (FR)

(21) Appl. No.: 18/689,404

(22) PCT Filed: Aug. 22, 2022
(86) PCT No.: PCT/FR2022/051596

§ 371 Date: Mar. 5, 2024

(30) Foreign Application Priority Data
Sep. 6, 2021 (FR) FR2109315

(51) Int. Cl. A01K 67/033; B65D 85/50

(52) U.S. Cl. CPC A01K 67/033; B65D 85/50

(57) ABSTRACT

A cage for the industrial production of insect eggs is made up of an enclosure delimited by a ceiling and four side walls defining a confined box-shaped space for the insects. The enclosure contains an area for accommodating at least one container of pupae and means for attaching and extracting collectors for collecting the eggs laid by the insects confined in the cage. The front wall of the enclosure has at least one first opening that can be closed by a first door for accessing the area for accommodating at least one container of pupae, a second door for enabling an operator to access the inside of the cage, and at least one opening lined with additional closure means for inserting and removing the egg collectors. The cage has a dark inner space that communicates with the first door and with the rest of the enclosure.

### CROSS REFERENCE TO RELATED APPLICATIONS

This application is a national phase entry under 35 U.S.C. § 371 of International Patent Application PCT/FR2022/051596, filed Aug. 22, 2022, designating the United States of America and published as International Patent Publication WO 2023/031536 A1 on Mar. 9, 2023, which claims the benefit under Article 8 of the Patent Cooperation Treaty to French Patent Application Serial No. FR2109315, filed Sep. 6, 2021.

### TECHNICAL FIELD

The present disclosure relates to the field of large-scale farming of arthropods and, in particular, of insects, in particular, of the black soldier fly, intended, for example, for the production, from insect larvae, of animal or human foods. It relates more particularly to equipment intended for the reproduction and mating step of the insects, starting with the introduction of the pupae and ending with the collection of the eggs, which are then transferred to another equipment item intended for the hatching step.

This reproduction and mating step is generally done inside cages allowing insects to have sufficient living space while remaining confined in an enclosed space. The climate conditions are controlled inside these cages in order to guarantee optimal conditions.

### BACKGROUND

French Patent No. FR3027488 discloses an insect farming unit wherein it comprises at least one closed chamber and a system for controlling the environmental conditions of the chamber. The closed chamber further comprises at least one upper compartment intended to contain insects, the upper compartment comprising at least one airlock, generally used to pass food; the closed chamber also comprises at least one lower compartment intended to receive the insect eggs contained in the upper compartment; the closed chamber further comprises at least one separating element for separating the upper and lower compartments, the separating element comprising pores; the upper and lower compartments are removable and separable from the chamber, independently of one another.

U.S. Pat. No. 10,667,502 is also known, disclosing a large-scale and multi-span breeding greenhouse for adult black soldier flies, comprising greenhouse bodies. Each greenhouse body comprises a pupae room and a breeding and oviposition room; a partition wall is provided between the pupae room and the breeding and oviposition room, the partition wall is provided with a door hole communicating the pupae room and the breeding and oviposition room; the pupae room is opaque; the greenhouse body further comprises a light guiding device that introduces sunlight into the breeding and oviposition room.

These solutions are not fully satisfactory because the introduction of the containers containing the pupae, the removal of the eggs and the maintenance operations are carried out in a space whose walls must prevent the flies from leaving their confined space. More generally, the solutions known in the art leave an excessive risk of flies escaping from the cages, in particular, during the introduction or extraction of the egg collectors.

Moreover, the solutions known in the art have a relatively complex geometry, with corners that can trap flies and make full cleaning difficult.

Moreover, the solutions known in the art have the disadvantage of multiplying the risks of eggs being laid outside the collectors due to their complex geometries having multiple corners. Indeed, it is known that flies tend to lay their eggs in corners.

Finally, the solutions known in the art are not optimal for cleaning or maintenance procedures in the absence of flies.

### BRIEF SUMMARY

In order to address these drawbacks, the present disclosure relates, in its most general sense, to a cage for the industrial production of insect eggs comprising an enclosure delimited by a ceiling and four side walls comprising a front wall, defining a confined box-shaped space for the insects, the enclosure containing an area for accommodating at least one container of pupae and means for attaching and extracting collectors for collecting the eggs laid by the insects confined in the cage. The front wall of the enclosure has: at least one first opening that can be closed by a first door for accessing the area for accommodating at least one container of pupae, a second door for enabling an operator to access the inside of the cage, and at least one opening lined with additional closure means for inserting and removing the egg collectors. The cage has an accommodating area constituting a dark inner space that communicates, on the one hand, with the first door and, on the other hand, with the rest of the enclosure.

Advantageously, the dark area has at least one side outlet oriented toward a light area of the cage.

According to one variant, one of the walls has at least one opening with a section adapted to the cross section of oviposition means.

Preferably, the junctions between two planar surfaces of the cage have a rounded shape. By avoiding sharp angles and corners, the rate of laying outside the collectors is reduced. These junctions mainly relate to the assembly of the walls. They also optionally relate to the connection of accessories to the walls.

The present disclosure also relates to a juxtaposition of cages according to at least one of the above elements.

### BRIEF DESCRIPTION OF THE DRAWINGS

Other features and advantages will become apparent from the following description of the invention, given only by way of example, referring to the accompanying drawings wherein:

FIG. 1 depicts a perspective view of an installation comprising cages according to the present disclosure; and

FIG. 2 depicts a perspective view of an opening lined with additional sealed closure means for the insertion and removal of egg collectors for a cage according to the present disclosure.

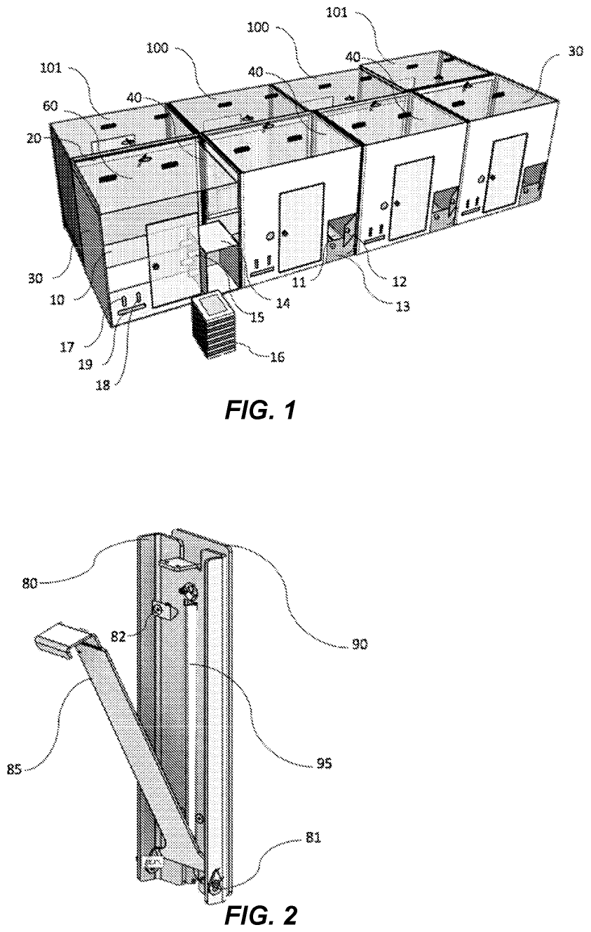

### DETAILED DESCRIPTION

The present disclosure relates to an insect breeding installation dedicated to the production of eggs, in the context of an industrial system for producing and transforming insects. Usually, such an installation comprises an egg production chamber configured to receive insect pupae that will end nymphosis in the chamber, and allow the emerging adult insects to mate and to lay eggs on collectors generally associated with an oviposition means to avoid the dispersion of laying sites. Collectors, for example, ribbed panels, constitute the laying areas to facilitate the clustering and collection of the eggs and their transport. These collectors are then moved into another installation intended for the maturation and storage of the eggs. Adult insects are often subjected to controlled illumination with at least one wavelength of light conducive to the breeding between insects. Besides the wavelength, the illumination intensity is also controlled during the day to be conducive to breeding by modulating this intensity and alternating the periods with and without illumination.

The simplicity of handling and cleaning is an essential issue to allow controlled industrial production. The installation comprises a group of cages (100, 101) having an open bottom installed in a building with climate controlled conditions, glazed for natural lighting, or opaque for artificial lighting. The building floor serves as lower closure of the cages (100, 101). The floor of the building may have discharge means for waste collection. Inside this building, only the temperature is regulated: the value of this temperature must be as close as possible to the value inside the cages while allowing acceptable working conditions for operators present in the building. A second system then allows finer control of the climate conditions (in particular, the humidity and the temperature) inside the cages.

Each cage (100, 101) is a light construction intended to define a box-shaped space for confining insects moving in the cage. The cages (100) located at the core of the group consist of a rigid front wall (10), for example, made of PLEXIGLAS®, two side walls (40) common to two neighboring cages, a rear wall (20) common to two neighboring cages and a ceiling (60). The cages (101) located at the ends of the group consist of two side walls (30, 40).

According to a particular embodiment, the rear wall (20) is made up of a mix of netting and rigid walls; rigid walls are provided facing the collectors in order to be able to attach the attaching devices thereto, the rest will be netting to allow air circulation. The side wall (30) is rigid and sealed because it delimits the group of cages.

Preferably, the cages form an open bottom structure, without a bottom, which are deposited on and attached to the floor of a building equipped, for example, with discharge means for waste collection.

The side wall (40) is made entirely of netting in order to allow air to circulate (apart from a small, rounded cornice at the bottom, at the top and on the sides to avoid corners).

In the example, the front wall (10) has an opening (11) provided with two doors (12, 13). This opening (11) opens into a dark and non-illuminated emergence airlock (14) formed inside the cage (100, 101), for example, by opaque walls. At least one of the walls is permeable to air in order to be able to place this emergence airlock under climate conditions. This airlock (14) communicates with the rest of the enclosure via ducts (15) enabling the flies to join the reproduction and laying space after emergence. The difference in brightness resulting from the opacity of the walls constituting the airlock (14) encourages the flies to leave the nymphosis area.

The doors (12, 13) make it possible to open the access to the airlock (14) to introduce at least one pupae container (16). The darkness in the airlock (14) means that few flies are present in the airlock (14), and the doors (12, 13) make it possible to introduce or remove a pupae container (16) without risk of escape. Optionally, lowering the temperature makes it possible to reduce the activity of the flies and therefore to minimize the risk of escape.

The front wall (10) also comprises an opening (19), advantageously provided with elastic sealing lips, intended to introduce a means of oviposition, for example, loaded with pheromones or a substrate, which can be placed under the laying collectors.

These laying collectors are attached to a support that is rigidly connected to the front (10) and rear (20) rigid walls and take the form of ribbed plates.

Openings (17, 18) also provided with mechanical barriers preventing any escape, or elastic sealing lips, make it possible to introduce and extract the collectors while preventing the insects from escaping from the cage during the handling operations of the collectors.

A door (15) allows an operator to enter the cage, in particular, for a repair or cleaning operation.

Preferably, the cages are juxtaposed to form a group, typically between 2 and 40 juxtaposed cages, preferably between 8 and 16 cages. The advantages relative to individual cages are related to managing climate conditions: a group of cages in fact makes it possible to significantly reduce HVAC (Heating, Ventilation and Air Conditioning) equipment, since the conditions are managed on the scale of the group of cages and not on the scale of the individual cage. This grouping therefore does not lead to any loss of precision (as long as the size of the group remains reasonable). Each cage in a given group of cages will have the same age and thus need the same climate conditions. This also has an advantage in terms of productivity, since all the cages reach the end of life at the same time: the stunning, the post-treatment of the flies and the cleaning can be done on the group of cages.

Example of Opening Lined with Complementary Sealing Means, for Insertion and Removal of Egg Collectors

FIG. 2 depicts an example of an opening lined with additional sealed closure means, for the insertion and removal of egg collectors.

It consists of the assembly of two folded sheets (or plastic parts) (80, 90) each having a slot (95) adapted to the insertion and extraction of an egg collector, that is, having a shape and dimensions determined based on the cross section of the egg collector in order to allow it to pass through the slot with a gap preventing the flies in the cage from being able to escape from the cage.

This slot (95) can be masked by a door (85) tilting about a transverse axis (81). Of course, the hinge can also be provided relative to a vertical axis. A mechanical solution like elastic, magnet, latch (82) ensures that the door (85) is held in a position pressed against the slot (95).

For a flexible cage wall formed by a cloth or netting, the two parts (80; 90) are positioned on both sides of a cut-out made in the wall, optionally braided to avoid tearing, and then clamped against one another by screws passing through the two parts (80, 90). The slot (95) made in the netting may be provided with a zipper on both sides of the door (85) to allow additional access.

The assembly is similar for rigid cage walls. In this case, a cut-out is made having a dimension greater than that of the slot (95) and less than the section of the parts (80, 90) in order to allow them to be positioned on both sides of the wall.

The sheet (80) can be attached by screwing, for example, on the front end of the guide rails of the egg collectors in order to ensure correct centering.

1. A cage for industrial production of insect eggs, comprising an enclosure delimited by a ceiling and four side walls including a front wall, ceiling and four side walls defining a confined box-shaped space for insects, the enclosure containing an area for accommodating at least one container of pupae and means for attaching and extracting egg collectors for collecting eggs laid by the insects confined in the cage, wherein, the front wall of the enclosure has:
at least one first opening that can be closed by a first door for accessing the area for accommodating at least one container of pupae,
a second door for enabling an operator to access the inside of the cage, and
at least one opening lined with additional closure means for inserting and removing the egg collectors; and
wherein the cage has an accommodating area constituting a dark inner space that communicates with the first door and with a remainder of the enclosure.

2. The cage of claim 1, wherein the dark inner space has at least one side outlet oriented toward a light area of the cage.

3. The cage of claim 1, wherein one of the walls has at least one opening with a section adapted to a cross section of an oviposition device.

4. The cage of claim 1, wherein junctions between two planar surfaces of the cage have a rounded shape.

5. An assembly for industrial production of insect eggs, comprising a plurality of cages according to claim 1 juxtaposed to one another.

6. A cage for industrial production of insect eggs, comprising:
an enclosure delimited by a ceiling and side walls defining a confined space for insects, the enclosure containing an area for accommodating at least one container of pupae, at least one of the side walls having at least one first opening adjacent to and communicating with the area for accommodating at least one container of pupae, at least one of the side walls having at least one second opening sized and configured to enable an operator to enter or exit the enclosure, at least one of the side walls having at least one third opening sized and configured for inserting and removing egg collectors for collecting eggs laid by the insects confined in the cage, the at least one third opening having a narrow elongated shape;
egg collectors for collecting eggs laid by the insects confined in the cage;
a first door configured to open and close the at least one first opening;
a second door configured to open and close the at least one second opening;
a closure mechanism covering the at least one third opening; and
interior walls within the enclosure delimiting an dark interior accommodating area within the enclosure in communication with the at least one first opening and with a remainder of the enclosure outside the dark interior accommodating area.

7. The cage of claim 6, wherein the dark interior accommodating area has at least one outlet oriented toward an illuminated area of the cage.

8. The cage of claim 7, wherein the illuminated area of the cage is a naturally illuminated area of the cage.

9. The cage of claim 6, wherein one of the walls has at least one opening with a section adapted to a cross section of an oviposition device.

10. The cage of claim 6, wherein junctions between side walls have a rounded shape.

11. An assembly for industrial production of insect eggs, comprising a plurality of cages according to claim 6 juxtaposed to one another.
